(19) United States
(12) Patent Application Publication
(10) Pub. No.: US 2013/0303979 A1
(43) Pub. Date: Nov. 14, 2013

(54) SUCTION AND IRRIGATION DEVICE

(71) Applicants: Lennart Henning Stieglitz, Muri bei Bern (CH); Andreas Spiegelberg, Buxtehude (DE)

(72) Inventors: Lennart Henning Stieglitz, Muri bei Bern (CH); Andreas Spiegelberg, Buxtehude (DE)

(73) Assignee: Universitat Bern Verwaltungsdirektion, Bern (CH)

(21) Appl. No.: 13/989,547

(22) PCT Filed: Nov. 24, 2011
(86) PCT No.: PCT/EP11/70952

§ 371 Date: Jul. 25, 2013

(30) Foreign Application Priority Data
Nov. 26, 2010 (EP) 101928240.

(51) Int. Cl. A61M 3/02

(52) U.S. Cl. CPC A61M 3/0283
USPC 604/30

(57) ABSTRACT

A suction and irrigation device for medical use comprises a valve body with an irrigation passage and a suction passage. At least one irrigation pipe and at least one suction pipe are connectable to the irrigation passage and the suction passage by connections at a proximal side of the valve body. A suction/irrigation tube is connectable to the irrigation passage and the suction passage at a distal side of the valve body by at least one further connection. A valve assembly is provided within the valve body, which in a first position opens the suction passage and closes the irrigation passage and in a second position closes the suction passage and opens the irrigation passage. The device comprises at least one push element which interacts with at least one movable element of the valve assembly, whereas the movable element is movable by mechanical force acted upon the push element such, that the push element activates the valve assembly from one position to the other position.

### CROSS-REFERENCE TO RELATED APPLICATIONS

This application claims priority to European Application No. 10192824.0 filed Nov. 26, 2010 and to PCT Application No. PCT/EP2011/070952 filed Nov. 24, 2011, all of which are incorporated herein by reference and made a part hereof.

### BACKGROUND OF THE INVENTION

1. Field of the Invention

The invention relates to a suction and irrigation device for medical use, in particular to a handheld suction and irrigation device operated by a medically trained person.

2. Description of the Related Art

Suction and irrigation devices are regularly used in several medical fields, for example in surgery, dentistry or general medical care. Such devices comprise a suction line and an irrigation line connected to a proximal side of a handhold, while a tip-hole is provided on a distal side, which is directed to the treatment area. The suction line is connected to a vacuum device to suck fluids through the tip-hole and the irrigation line is connected to a fluid reservoir to direct an irrigation fluid to the treatment area via the tip-hole. The handhold may comprise an actuation unit in form of switch or valve for switching from a suction modus to an irrigation modus and back. Many methods of treatment require repeated back and forth switching between suction and irrigation. Therefore the actuation unit has to be easily accessible and operable.

In EP 0 553 461 A1 (Elektronik-Vertrieb GmbH), a conventional suction and irrigation device is shown. It includes a handpiece to which an irrigation tubing and a suction tubing are connected, and a tubular extension piece. The tubular extension piece comprises an irrigation channel and a suction channel and is introduced into a surgical wound. The suction channel of the handpiece communicates with the surrounding atmosphere via a bypass bore. By closing the bypass bore, fluid is drawn off. The irrigation fluid is delivered by means of a roller pump which is activated by a foot switch permitting a precise metering of the delivery rate. In addition to a volume control of the irrigation pump a pressure control can also be provided, for which purpose the irrigation fluid line is provided with an irrigation clamp. The irrigation clamp opens or closes an irrigation tube running through the handpiece. The irrigation and suction device permits a controlled fluid environment in a surgical wound, as a result of which formation of pools or drying-out of the tissue is prevented. The surgeon can operate the device with one hand by opening and closing the bypass.

U.S. Pat. No. 4,680,026 (G. Deane, B. O. Weightman) discloses a hand-held suction-irrigator for surgical use, which has a handle and two inlet bores extending through the handle and connected respectively to a suction source and a source of irrigating fluid. A valve assembly connects one or the other of the bores to a single outlet port so that the outlet port can be used to apply suction or irrigating fluid. A probe is removably coupled to the outlet port by cooperating luer fittings. The two inlet bores are parallel with one another, the outlet port being located intermediate the inlet bores on the opposite side of the valve assembly. A valve member is mounted in the valve assembly and can be displaced transversely of the bores by pushing down on the valve member with a finger or thumb which have to be placed exactly on the valve member. When pushing the valve member a spring member is cocked which returns the valve member to the previous position after the valve member is released.

WO 2009/019538 A1, which is equivalent to U.S. Patent Publication No. 2011/0230823, (Lina Medical APS) discloses a valve mechanism for a suction and irrigation instrument. It comprises a first tubular housing provided with a first distal conduit for coupling to the instrument, and conduit means for communicating with a suction/irrigation source comprising a first and a second proximal conduit and a first piston movably arranged in the first tubular housing between a first position and a second position. The first piston has a first through-opening intersecting the longitudinal axis of the first piston. Upon movement of the first piston into the first position the first through-opening opens a pathway between the first distal conduit and the first proximal conduit, and upon movement of the first piston into the second position the first through-opening opens a pathway through the first distal conduit and the second proximal conduit.

Known suction and irrigation devices need high fine motor skills to switch between suction and irrigation modus, while simultaneously other treatments have to be completed. In practice often two hands are used to switch the device from suction to irrigation to ensure safe switch-over. In same cases a lot of force is needed to actuate the switch or valve which may lead to shivering of the hand and therefore of the suction and irrigation device. This can disturb or interrupt the treatment and even increase surgical risk, if the instrument is used together with a surgical microscope and therefore small movements of the instrument may lead to unwanted excursions in microscopical view.

It is an object of the present invention to provide a suction and irrigation device and a valve body for such a device, which enable simple manual switching between a suction position and an irrigation position and provide simple and uncomplicated preparation of the suction and irrigation device.

### SUMMARY OF THE INVENTION

These and other objects are solved by a suction and irrigation device according to claim 1. Other embodiments are described in dependent claims.

A suction and irrigation device for medical use according to the invention comprises a valve body with an irrigation passage and a suction passage, at least one irrigation pipe, at least one suction pipe and a suction/irrigation tube. The irrigation pipe and the suction pipe are connectable to the irrigation passage and the suction passage by connections at a proximal side of the valve body. The suction/irrigation tube is connectable to the irrigation passage and the suction passage at a distal side of the valve body by at least one further connection. The valve body comprises a valve assembly, which in a first position opens the suction passage and closes the irrigation passage and in a second position closes the suction passage and opens the irrigation passage. The valve assembly is located within in the valve body and comprises at least one movable element. The device further comprises at least one push element, which interacts with the at least one movable element. By mechanical force acted upon the push element, the at least one movable element is moved such that the valve assembly is activated from one position to the other position. That means the push element may be pushed from an initial position to an operated position, in which the push element actuates the valve assembly. When releasing the push force, the push element preferably returns back to its initial position.

The suction and irrigation device according to the invention can be operated easily from a first position to a second position and back with one hand without the need of changing finger positions on the device or even to find a grip for holding the device. Thus the suction device can be kept still during surgery and other applications. Advantageously, the valve body is simple to be connected to the suction and irrigation pipes of the device.

Preferably, in a state where no mechanical force is acted upon the push element the movable element is held by elastic forces in a position where the irrigation passage is closed and where the suction passage is open. This allows for a simple operation of the device, where essentially the user presses the push element for irrigation. As soon as the push element is released, the device will switch back to suction. Furthermore, the use of elastic forces provides for having a defined default position and simplifies the construction of the device.

In a preferred embodiment, the push element is a push surface comprised by the valve body and the movable element is an elastically deformable element, which is deformable by mechanical force acted upon the push surface.

In the context of this embodiment, preferably the at least one push surface is elastically deformable, in particular the valve body comprises a large area of an elastically deformable push surface on one side. For example the large area comprises the size of a finger tip or a thumb tip. Basically the complete surface of one side of the valve body may be designed as elastically deformable push surface. The elastically deformable push surface preferably extends over the irrigation passage and the suction passage within the valve body.

Advantageously, a second push surface, arranged opposite to the first push surface is provided such, that the first and the second push surfaces include the valve assembly in between each other. Thus the valve assembly can be actuated by pressing both sides of the valve body. The push force can be applied from a top side and a bottom side of the body for example. Again, the push surfaces are preferably elastically deformable. Preferentially lateral conjunction surfaces or walls for conjoining top and bottom surfaces are designed less elastically deformable as the at least one push surface. Then the lateral conjunction surfaces stabilize the valve body.

The valve body can for example be designed of two mold components which when put together form the valve assembly. Preferably the top side and the bottom side each are produced as one mold component. These two components are fixed together along the conjunction surfaces and together built the valve assembly in the interior of the two components. Thus the valve body can be produced in a cheap and simple mannerner.

Preferably, at least the push surface of the valve body is of an elastomeric material, in particular a thermoplastic elastomer or a silicone elastomer. Most preferably, in the case of a valve body designed of two mold components forming the valve assembly, both mold components are made of such a material.

The valve body itself may serve as a housing for the valve assembly. Alternatively the valve body is carried by a strong and rigid housing comprising at least one opening through which the at least one push surface of the valve body is accessible. Such the at least one push surface can be activated through the opening. By using a separate housing also very soft materials may be used for the valve body.

The valve assembly preferably comprises a first valve within the suction passage and a second valve within the irrigation passage, which are commonly deformable by the push surface. Therefore the irrigation passage and the suction passage are provided close to each other such, that the push surface can be operated by the mechanical force of one finger tip and interact simultaneously with both valves.

In one embodiment the first valve is designed by at least one first closing ledge on an inner wall of the suction passage and an opposing inner surface of the valve body. Alternative to the opposing surface of the body at least one second closing ledge may be provided on an opposing inner wall of the valve body. Preferably at least one of the closing ledges is arranged on an inner wall of the at least one push surface. In the first position of the valve assembly exists a channel between the first closing ledge and the opposing inner surface or the second closing ledge respectively. In the second position the first closing ledge and the opposing inner surface or the second closing ledge contact each other and form a fluid tight closure, when the push surface is pressed. The elasticity of the valve body material assists to create a fluid tight contact of the two ledges or the ledge and the inner wall of the valve body. Even small grooves or slits can be closed by the deformation of the material. Preferably such a valve design is used in the suction passage.

Advantageously, the second valve comprises at least two elastically deformable lamellas, which project from opposing lateral inner walls and are aligned essentially perpendicular to the push surface. For example the lamellas are provided on a lateral conjunction surface and an internal partition wall, which separates the suction passage from the irrigation passage. Such a valve design is used in the irrigation passage preferably. The front edges of the lamellas can contact each other and form a fluid tight closure in the first position. Basically between the two lamellas there is provided a slit running perpendicular to the push surface and which is fluid tight closed in the first position. In the second deformed position the lamellas form a channel, when the push surface is pressed. Advantageously the elastically deformable lamellas are deformable about two different deformation axes lying essentially perpendicular to each other. One deformation axis may run perpendicular to an axial direction of the irrigation passage and/or perpendicular to the push surface. The other deformation axis may run parallel to the push surface. Therefore the lamellas can be bent in axial direction of the irrigation passage towards lateral walls of the valve body. Also they can be curved in direction of the large area surfaces of the valve body, which leads to curved edges of the tips. By deformation of the lamellas an opening in the middle part of their edges in between them is created. In this embodiment the at least one push surface extends over the at least one closing ledge of the first valve and the lamellas of the second valve.

Between the suction passage and the irrigation passage an internal partition wall is provided within the valve body. The internal partition wall may comprise two parallel walls with an intermediate cavity in between them. Such the internal partition wall in fact is built of two lateral surfaces of each of the passages. When pressing the push surface the ledge and the lamellas of the valve body and the partition wall are deformed. The intermediate cavity can compensate the differing deformation of the lateral surfaces of each passage and assist the valve assembly to work properly. The intermediate cavity allows sufficient individual flexibility that each lateral surface can be deformed according to the needs of the different valves, because each internal walls of the partition wall can be deformed individually.

In a preferred embodiment the suction and irrigation device according to the invention comprises an opening, which opens to the suction passage. Preferably, the opening is arranged in the push surface. By opening, closing or partly covering the opening with a finger tip the suction effect in the suction passage can be controlled simultaneously to actuating the valve assembly. The general operation of an opening to the suction passage is known from the prior art.

Alternatively, the opening to the suction passage is not arranged in the push surface but at another place, e.g. neighboring the push surface or a push element. In principle, the opening may open to the suction passage (i.e. downstream of the valve) or to the passage upstream of the valve.

Mostly the suction passage and the irrigation passage end in a common distal outlet tube serving as suction/irrigation tube and comprising a distal tip, which is positioned at the operation area. That means the distal tube serves both for suction and irrigation depending on the position of the valve assembly. In the first position, when the suction valve in the suction passage is open, the distal tube acts as a suction channel. In the second position, when the irrigation valve in the irrigation passage is open, the distal tube acts as an irrigation channel.

In another embodiment the suction and irrigation device comprises separate distal channels for suction and irrigation, which can be provided in a common or separate distal tube. In this case the device can suck and flush the operation area simultaneously by pressing the push surface to a middle position between first and second position. By pressing the push surface only slightly, the suction valve with the closing ledge is not yet completely closed while the irrigation valve with the lamellas already started to open. That means both valves of the valve assembly are open at the same time and irrigation medium can flow to the operation area and liquid can be removed thereof.

Instead of pressing the push surface directly by finger or thumb the housing, which encompasses the valve body may comprise a lever arrangement or a screw arrangement. The lever arrangement can enlarge manual force to facilitate the compression of the valve body. The screw arrangement may comprise a screw, which is located in a threaded opening within the housing. By screwing the screw into the opening of the housing the inner end of the screw may apply a push force on the push surface.

Instead of having a push surface comprised by the valve body, the device may comprise a push element which is separate from the valve body, such as a push button or lever. Furthermore, the movable element may be a rigid element allowing for alternately closing the irrigation or the suction passage, respectively. In this case, the movable element, when acted upon by the push element, moves in such a way that the irrigation passage is opened whereas the suction passage is closed.

Instead of having elastically deformable elements of the valve body, the elastic forces may be exerted by separate elements such as a spring.

In further preferred embodiments, the movable element is ferromagnetic and the elastic forces are exerted by a magnetic field. As an example, the movable element may be a ball made from a ferromagnetic material, the size of which being adapted to the cross section of the irrigation and suction passages.

In this context, the irrigation passage is preferably provided with at least one ferromagnetic element, preferably with a ring surrounding the passage. Especially preferred is a ring having a circular outer shape. Either this ferromagnetic element or the movable element or both are permanently magnetized, such that there is an attractive force between these two components. In the case of having a ring surrounding the passage and if the magnetic ring cooperates with a ball made from ferromagnetic material, the diameter of the ferromagnetic ring preferably is in the range of 0.5-2 times the diameter of the magnetic ball, in particular in the range of 0.8-1.2 times the diameter of the magnetic ball.

However, instead of the ring the irrigation passage may be provided with a single magnetic element which does not or only partially surround the passage, or with a plurality of magnetic elements such as segments, the totality of them surrounding the passage. Alternatively or in addition, the movable element may as well be permanently magnetized. In a further alternative, the irrigation passage is provided with a ferromagnetic but essentially not magnetized element, and just the movable element (such as a ball) is permanently magnetized.

Using magnetic forces avoids the need for mechanical elements such as springs or dedicated elastic regions of the valve body. Accordingly, the construction may be simplified and/or the durability of the device is enhanced.

Preferably, upon acting a mechanical force upon the push element the movable magnetic element is moved in a direction parallel to a wall including the irrigation passage until it covers the suction passage. For that purpose, the angle between the axes of the passages is preferably in the range of 80-100°, most preferably 90°. This allows for a simple construction and ensures reliable interaction between the magnetic element and the mouths of the passages being contacted by the movable element.

These and other objects and advantages of the invention will be apparent from the following description, the accompanying drawings and the appended claims.

### BRIEF DESCRIPTION OF THE DRAWINGS

Preferred embodiments of the invention will be described in the accompanying drawings, which may explain the principles of the invention but shall not limit the scope of the invention. The drawings illustrate:

FIG. 1 is a 3-dimensional view of a first embodiment of a suction and irrigation device according to the invention;

FIG. 2 is a cross-sectional view of a valve body according to the invention;

FIG. 3A is a schematical view of a first valve of a valve assembly in an open position;

FIG. 3B is a schematical view of the first valve in a closed position;

FIG. 4A is a schematical view of a second valve of the valve assembly in a closed position;

FIG. 4B is a schematical view of the second valve in an open position;

FIG. 5A is a schematical illustration of the operation of a suction and irrigation device according to the invention in a first valve position;

FIG. 5B is a schematical illustration of the operation of the suction and irrigation device in an opened first position;

FIG. 5C is a schematical illustration of the operation of the suction and irrigation device in a second valve position;

FIGS. 6A-6D are schematical illustrations of a valve body and a cover of a second embodiment of a suction and irrigation device according to the invention;

FIGS. 7A, 7B are schematical illustrations of the valves in their suction and irrigation position, respectively;

FIGS. 8A-8E are schematical illustrations of a valve body and a cover of a third embodiment of a suction and irrigation device according to the invention; and

FIGS. 9A, 9B are schematical illustrations of the valves in their suction and irrigation position, respectively.

### DETAILED DESCRIPTION OF THE PREFERRED EMBODIMENTS

In general in the figures same elements are indicated with same reference numbers.

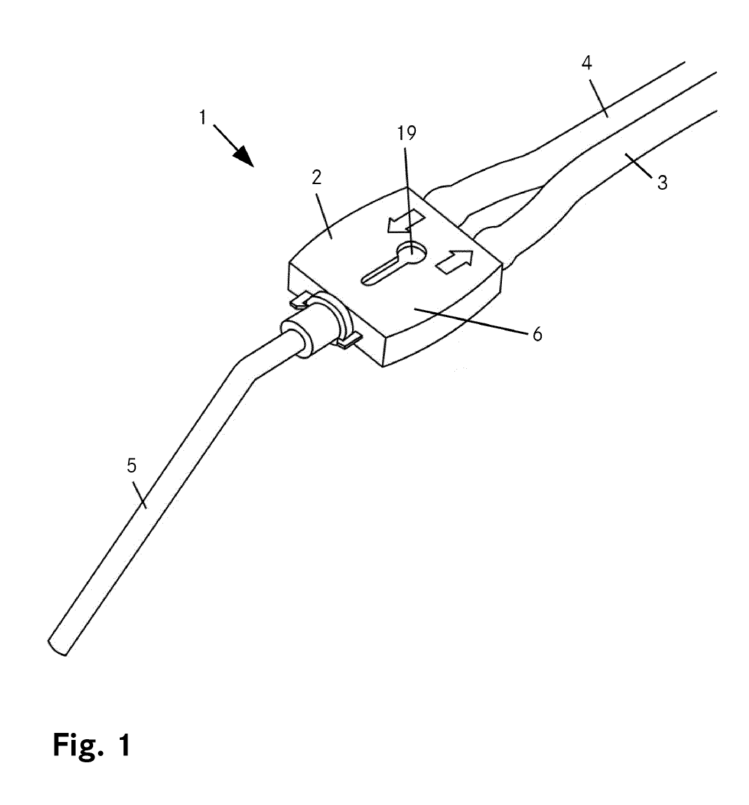

In FIG. 1, a first embodiment of a suction and irrigation device 1 is shown comprising a valve body 2 with a connected proximal irrigation pipe 3, a connected proximal suction pipe 4 and a distally connected suction/irrigation tube 5. The irrigation pipe 3, the suction pipe 4 and the suction/irrigation tube 5 are attached to the valve body 2 by connectors, for example a Luer lock or similar port. The valve body 2 is designed as a rectangular body comprising a large area at the top and the bottom and four lateral surfaces connecting top and bottom. The large top area is designed as an elastically deformable push surface 6 according to the invention. The distal lateral surface comprises the distally connected suction/irrigation tube 5 and the proximal lateral surface comprises the proximally connected irrigation and suction pipes 3 and 4. The size of the large area and the push surface 6 respectively is about 2×2 cm and the height of the lateral surfaces is about 0.5 cm. The valve body is made of silicone and the thickness of the push surface 6 is adapted to create a resiliently deformable surface.

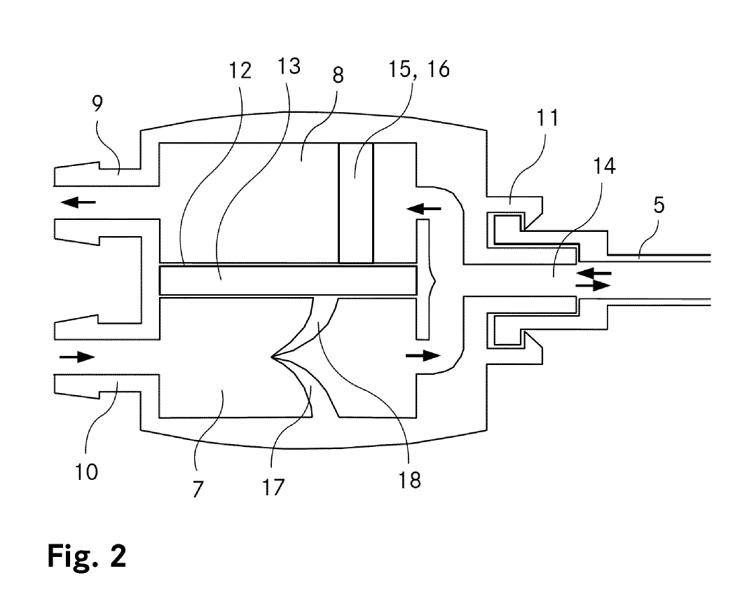

In FIG. 2 a cross sectional view of the interior of the valve body 2 is shown. The valve body 2 comprises an irrigation passage 7 and a suction passage 8 running between a first connector 9 for the suction pipe 4 and a second connector 10 for the irrigation pipe 3 on the proximal side and a distal connector 11 for the suction/irrigation tube 5 on the distal side. The irrigation passage 7 and the suction passage 8 are separated within the valve body 2 by a partition wall 12 comprising an intermediate cavity 13. Near the distal connector 11 irrigation passage 7 and suction passage 8 join to build a common exit channel 14 leading to the suction/irrigation tube 5.

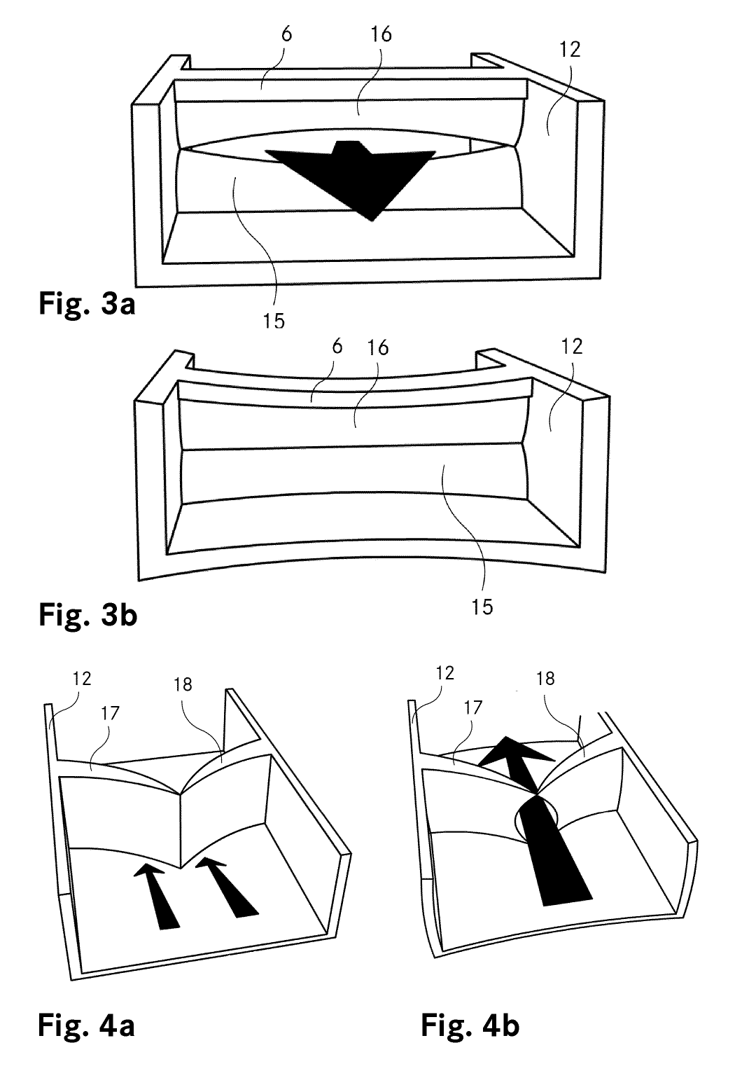

The suction passage 8 comprises a first closing ledge 15 arranged on the inner side of the large area surface of the valve body 2. Opposite to the first closing ledge 15 a second closing ledge 16 is arranged on the inner side of the push surface 6 as can be seen in FIGS. 3A and 3B. The first and the second ledges 15 and 16 together form a first valve of a valve assembly arranged within the valve body 2. The irrigation passage 7 comprises two elastically deformable lamellas 17 and 18, wherein the first lamella 17 is arranged on an inner side of a lateral wall of the valve body 2 and the second lamella 18 is arranged on an inner side of the partition wall 12. The lamellas 17 and 18 stretch out into the irrigation passage 7 such that their respective tips contact each other. The lamellas 17 and 18 together form a second valve of the valve assembly arranged within the valve body 2. The push surface 6 extends over the first and second valves of the valve assembly form one lateral wall to the opposing lateral wall of the valve body 2 covering the closing ledges 15 and 16 as well as the lamellas 17 and 18. The push surface 6 comprises an opening 19 towards the suction passage 8 as can be seen in FIG. 1.

In FIGS. 3A and 4A the valves of the valve assembly are shown in a first position corresponding to an initial position without pushing the push surface 6. In the first position the valve of the suction passage is open and the valve of the irrigation passage is closed. In FIGS. 3B and 4B the valves of the valve assembly are shown in a second position corresponding to a push position of the push surface 6. In the second position the valve of the suction passage is closed and the valve of the irrigation passage is open.

In the first position a channel exists between the first and the second closing ledges 15 and 16 as shown in FIG. 3A. That means the valve of the suction passage 8 is open. When the push surface 6 is pressed and compresses the valve body 2 into the second position, the distance between the top and the bottom surface of the valve body 2 is reduced and the first closing ledge 15 contacts the second closing ledge 16 as can be seen in FIG. 3B. That means the channel between the ledges 15 and 16 is closed and therefore the valve of the suction passage 8 is closed.

Simultaneously the lamellas 17 and 18 close the irrigation passage 7 in the first initial position as shown in FIG. 4A. The tips of the lamellas 17 and 18 contact each other and therefore the valve of the irrigation passage 7 is closed. The lamellas 17 and 18 are angled or slightly twisted about an axis perpendicular to the axis of the push surface 6 towards the incoming flow of irrigation fluid. Thus the pressure of irrigation flow presses against the lamellas and forces them into the closing position. When narrowing the distance between the large surfaces of the valve body 2 by pushing the push surface 6 the lamellas 17 and 18 are deformed about an axis parallel to the push surface 6 such that the tips of the lamellas are curved in a concave shape and encompass a channel in between each other. That means the valve of the irrigation passage 7 is open and irrigation fluid can pass through the passage. By pushing the push surface 6 the lateral wall and the opposing partition wall 12 are slightly deformed as well. To guarantee that the deformation of the partition wall 12 caused by opening the lamella valve does not disturb fluid tight closing of the ledge valve, the intermediate cavity 13 may compensate the difference of deformation of the inner partition wall of the suction passage 8 and the inner partition wall of the irrigation passage 7.

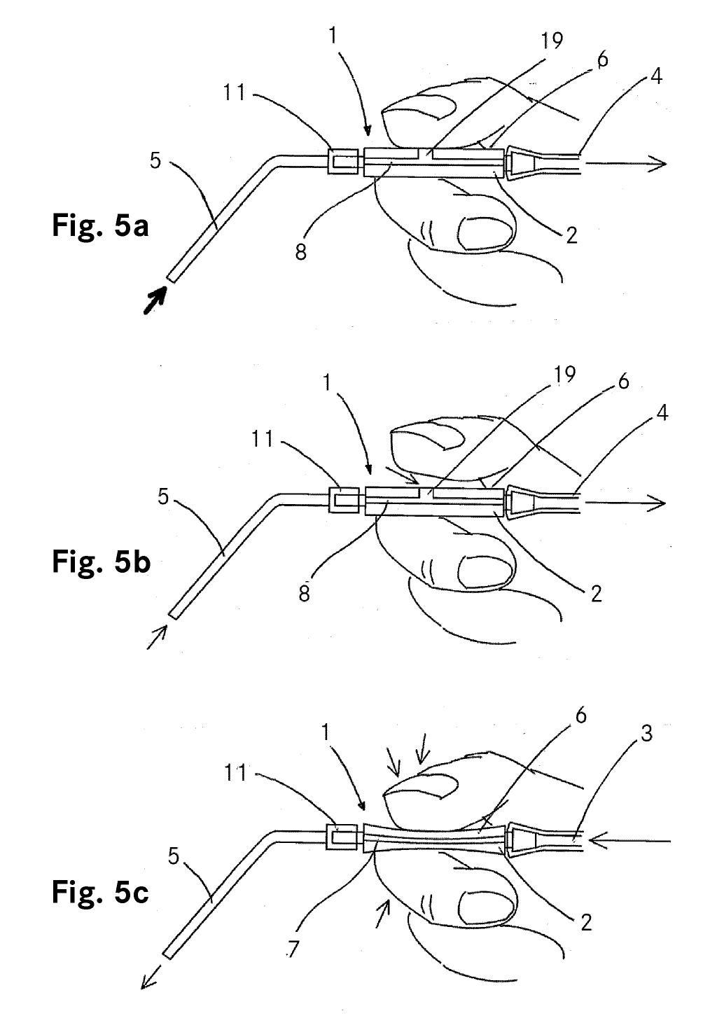

In FIG. 5A to 5C the handling of the suction and irrigation device 1 according to the first embodiment is explained. FIG. 5A shows the suction and irrigation device 1 with a schematical illustration of the suction passage 8 inside the valve body 2. The suction and irrigation device 1 is in a first initial position, wherein the suction passage 8 is open and the irrigation passage 7 is closed. The suction/irrigation tube 5 can be placed at an operation area, wherein liquid is supposed to be drawn off. The suction and irrigation device 1 is held between a thumb and a forefinger, while the thumb closes the opening 19 to the suction passage 8. The valve body 2 is not compressed in this position. A suction apparatus is connected to the suction pipe, which applies a suction force at the tip of the suction/irrigation tube. Since the opening 19 is closed full power of the suction force is generated at the tip and a strong suction effect acts on the liquid in the operation area.

To reduce the suction effect in the suction/irrigation tube 5, the opening 19 in the push surface 6 can be partially or completely opened by lifting the thumb as shown in FIG. 5B. Such the suction passage 8 is open to the atmosphere and the suction apparatus partly sucks air from the atmosphere. Therefore the suction effect at the tip of the suction/irrigation tube 5 is reduced.

In FIG. 5C the suction and irrigation device 1 is shown with a schematical illustration of the irrigation passage 7 inside the valve body 2 for explanatory reasons although the device does not have to be turned between the fingers. The suction and irrigation device 1 is in the second and compressed position, wherein the push surface 6 is impressed by the thumb to apply a compression force on the valve assembly within the valve body 2. That means the suction passage 8 is closed and the irrigation passage 7 is opened. The irrigation pipe 3 is connected to an irrigation apparatus providing irrigation fluid for the suction and irrigation device 1. The irrigation fluid flows through the open irrigation passage 7 and the suction/irrigation tube 5 to the operation area and flushes the area. By releasing the pressure on the push surface 6 the resilient material of the push surface 6 returns to the initial position for opening the suction passage 8 and closing the irrigation passage 7. Now the irrigation fluid and other liquids may be sucked through the suction/irrigation tube 5.

Advantageously it is not necessary take off the fingers from the suction and irrigation device 1 to switch between first and second position of the valve assembly. Thumb and forefinger may stay in place while only the push force between the fingers has to be adapted to change between suction and irrigation position.

As described before a housing may be provided for encasing the valve body. The push surface can remain accessible for applying a push force and switching valve positions within the housing. Or the housing may comprise additional push elements to push impress the push surface within the housing. In any case a simple actuation of the valve assembly is realized.

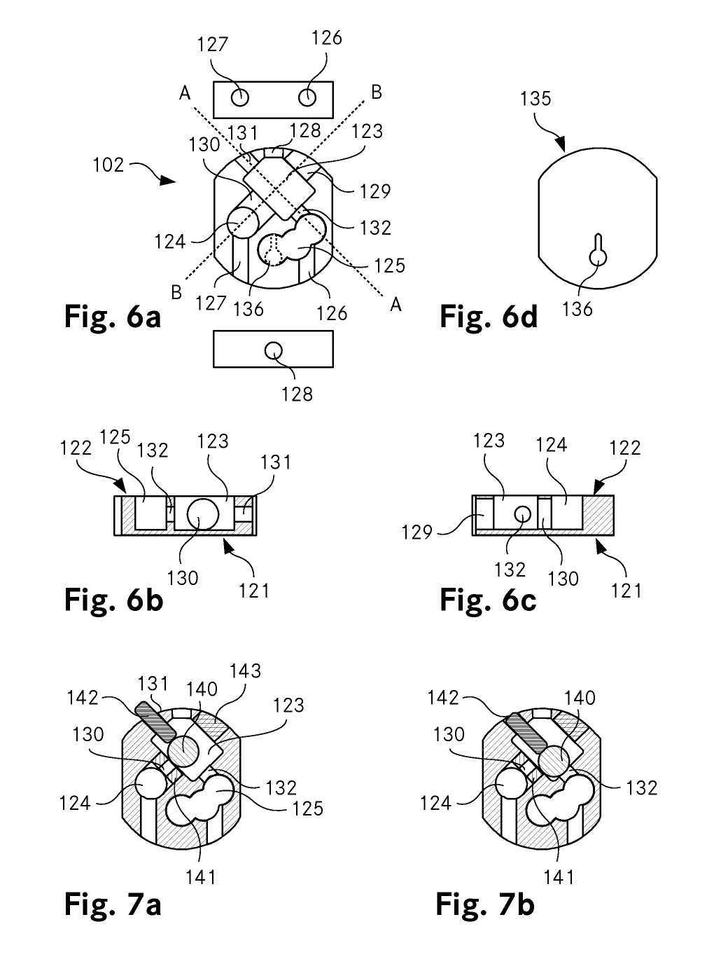

The FIGS. 6A-6C are schematical illustrations of a valve body of a second embodiment of a suction and irrigation device according to the invention. The FIG. 6A shows a cross-sectional view in a plane at about half the height of the valve body and extending parallel to the top surface as well as two views onto the front and back surfaces of the valve body. The FIG. 6B shows a cross-section along the plane A-A perpendicular to the top surface. The FIG. 6C shows a cross-section along the plane B-B perpendicular to the top surface as well as to the cross-section of FIG. 6B. The FIG. 6D shows a cover for the valve body.

The valve body 102 is made from PVC. Its footprint is oblong with straight lateral edges and rounded front (distal) and back (proximal) edges. The base surface 121 of the valve body 102 is continuous over the entire footprint. The top surface 122 features a number of blind hole like openings, namely a valve compartment 123 having a rectangular footprint, arranged close to the front edge (distal edge) of the valve body 102, angled 45° with respect to the longitudinal axis of the valve body 102. Further, the top surface 122 features an irrigation compartment 124 having a circular footprint and a suction compartment 125 having an elongated, substantially arc-shaped footprint. Both the irrigation compartment 124 and the suction compartment 125 are arranged proximally with respect to the valve compartment 123.

The valve body 102 further comprises a number of bores extending parallel to the base and top surfaces 121, 122, all having a circular cross section. Three of the bores are oriented in parallel to the longitudinal axis of the valve body 102, a first of them, the suction bore 126 leads from the back edge (proximal edge) to the suction compartment 125. A second of them, the irrigation bore 127 leads from the back edge (proximal edge) to the irrigation compartment 124. The third of them, the exit bore 128 leads from the front edge (distal edge) to the valve compartment 123.

There is a further bore 129 leading in a 45° angle from the front edge (distal edge) to the valve compartment 123. During manufacture of the valve body 102, this bore 129 is used for creating a connection bore 130 in the extension of the bore 129. The connection bore 130 connects the valve compartment 123 with the irrigation compartment 124. A further bore 131 extends in a direction perpendicular to the bore 129 and the connection bore 130 from the front edge (distal edge) to the valve compartment 123, and finally a connection bore 132 in the extension of this further bore 131 connects the valve compartment 123 and the suction compartment 125. The valve body 102 may be easily manufactured from a solid block just featuring the cuboid valve compartment by drilling 9 bores (four perpendicular to the top surface 122, five parallel to the top surface 122).

In the assembled state, the valve body 102 is covered by a cover 135 made from PVC, matching the footprint of the valve body 102, featuring a keyhole shaped opening 136 which connects the suction compartment 125 to the exterior. The length of the valve body 102 with attached cover 135 is about 30 mm, the width is about 25 mm, the thickness is about 8 mm

The FIG. 7A, 7B are schematical illustrations of the valves in their suction and irrigation position, respectively, showing the same cross section as FIG. 6A.

The bore 129 used for the manufacture is filled and sealed by a corresponding plug 143. A ball 140 made from a ferromagnetic material (such as stainless steel) is inserted into the valve compartment 123. A permanently magnetized ring 141 is accommodated within the connection bore 130 connecting the valve compartment 123 and the irrigation compartment 124. For that purpose, the diameter of the connection bore 130 is chosen to be larger than that of the other connection bore 132 and the bores leading to the front and back edges, the inside diameter of the magnetized ring 141 corresponds to the diameter of these further bores. Further, a pushing rod 142 made from PEEK is held within the bore 131 leading from the front edge (distal edge) to the valve compartment 123 and extending in the same axis as the connection bore 132 connecting the valve compartment 123 and the suction compartment 125.

Usually, the ball 140 is attracted to the permanently magnetized ring 141 and thereby closes the connection bore 130 connecting the valve compartment 123 and the irrigation compartment 124, i.e. the irrigation passage is closed. The inner edge of the permanently magnetized ring 141 constitutes a valve seat for the ball 140. At the same time, the suction compartment 125 is connected to the valve compartment 123 by the connection bore 132, i.e. the suction passage is open.

By pushing in the pushing rod 142 the ball 140 may be moved to engage the mouth of the connection bore 132 adjacent to the valve compartment 123, the inner edge of the connection bore 132 constituting another valve seat for the ball 140. In this second position, the suction passage is closed but the irrigation passage is open.

The handling of the suction and irrigation device according to the second embodiment essentially corresponds to the handling of the first embodiment explained before. As mentioned, as long as the pushing rod 142 is not operated, the suction and irrigation device is in a first initial position, wherein the suction passage is open and the irrigation passage is closed. The suction/irrigation tube connected to the exit bore 128 can be placed at an operation area, wherein liquid is supposed to be drawn off. The valve body 102 is held between a thumb and a forefinger, while the thumb closes the opening 136 to the suction compartment 125. A suction apparatus is connected to the suction bore 126 by means of a suitable connector. The suction apparatus applies a suction force at the tip of the suction/irrigation tube. Since the opening 136 is closed full power of the suction force is generated at the tip and a strong suction effect acts on the liquid in the operation area.

To reduce the suction effect in the suction/irrigation tube, the opening 136 in the cover 135 of the valve body 102 can be partially or completely opened by lifting the thumb. Such the suction compartment 125 is open to the atmosphere and the suction apparatus partly sucks air from the atmosphere. Therefore the suction effect at the tip of the suction/irrigation tube is reduced.

By pushing the pushing rod 142, e.g. with an index finger, the suction and irrigation device may be brought to the second position, where the suction passage is closed and the irrigation passage is opened. The irrigation bore 127 is connected to an irrigation apparatus by a suitable connector, the apparatus providing irrigation fluid for the suction and irrigation device. The irrigation fluid flows through the open irrigation passage and the suction/irrigation tube to the operation area and flushes the area. By releasing the pushing rod 142 the magnetic forces attracting the ball 140 will move the ball 140 to the magnetic ring 141, thereby closing the connection bore 130 to the irrigation compartment 124. At the same time, by the movement of the ball 140 the pushing rod 142 is moved in an outside direction. Movement of the pushing rod 142 in this direction may be restricted by a small collar resting on the area surrounding the inner mouth of the bore 131 accommodating the pushing rod 142. The resetting motion of the pushing rod 142 may be supported by an elastic element such as a spring arranged in between a further outside collar of the pushing rod 142 and the outer surface of the valve body 102.

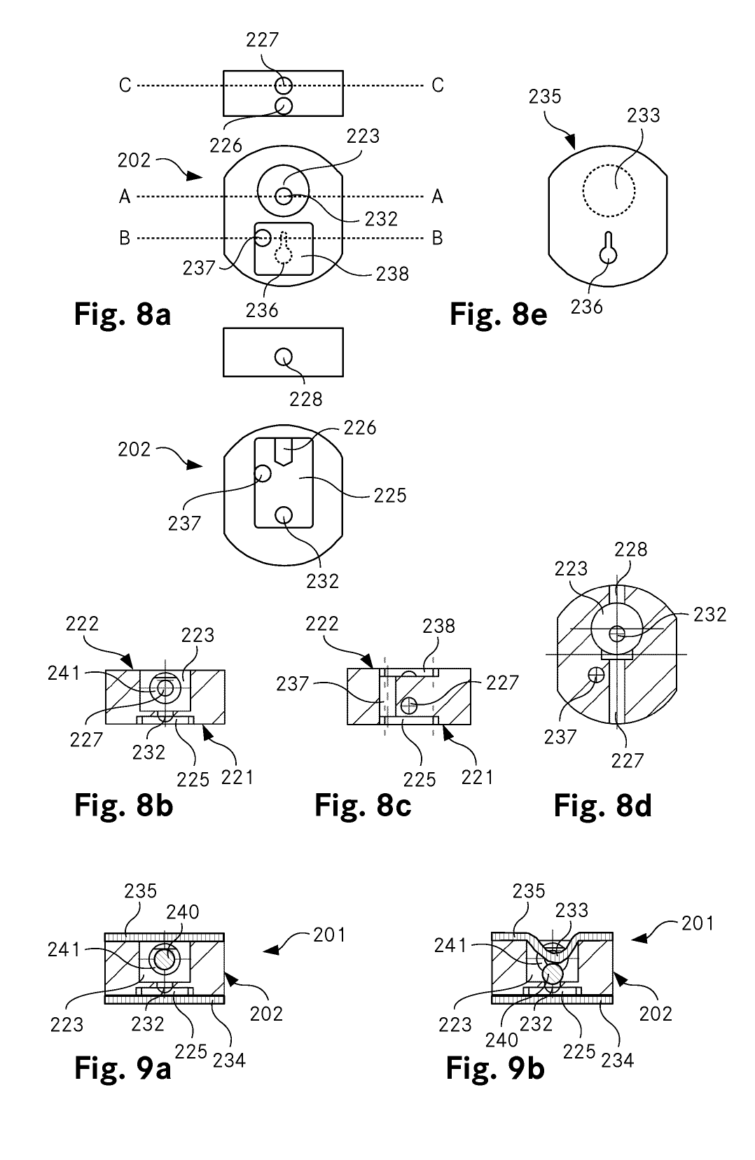

The FIG. 8A-8D are schematical illustrations of a valve body of a third embodiment of a suction and irrigation device according to the invention. The FIG. 8A shows a top view as well as two views onto the front and back surfaces of the valve body. The FIG. 8B shows a cross-section in a plane perpendicular to the top surface of the valve body, extending along A-A, the FIG. 8C shows a cross-section in a plane parallel thereto, along B-B. The FIG. 8D shows a cross-section in a plane parallel to the top surface along C-C. The FIG. 8E shows a cover for the valve body.

The valve body 202 is made from PVC. Its footprint is oblong with straight lateral edges and rounded front (distal) and back (proximal) edges. The base surface 221 of the valve body features a blind hole like opening, namely a suction compartment 225, having a substantially rectangular footprint. The top surface 222 features two blind hole like openings, namely a valve compartment 223 having a circular footprint, arranged close to the front edge (distal edge) of the valve body 202, the center of the circle arranged on the longitudinal symmetry axis of the valve body 202. Further, the top surface 222 features a suction control compartment 238 having a substantially quadratic footprint. The suction control compartment 238 is arranged proximally with respect to the valve compartment 223 and symmetric with respect to the longitudinal axis of the valve body 202.

The valve body 202 further comprises a number of bores extending parallel to the base and top surfaces 221, 222, all having a circular cross section. Three of the bores are oriented in parallel to the longitudinal axis of the valve body 202, a first of them, the suction bore 226 leads from the back edge (proximal edge) to the suction compartment 225. A second of them, the irrigation bore 227 leads from the back edge (proximal edge) to the valve compartment 223. The third of them, the exit bore 228 leads from the front edge (distal edge) to the valve compartment 223.

There are two further bores extending in a perpendicular direction with respect to the base surface 221 and the top surface 222 of the valve body, namely the connection bore 232 connecting the valve compartment 223 and the suction compartment 225 and the connection bore 237 connecting the suction compartment 225 and the suction control compartment 238, respectively. The connection bore 232 connecting the valve compartment 223 and the suction compartment 225 is arranged on the longitudinal axis of the valve body 202, in the center of the valve compartment 223. The other connection bore 237, connecting the suction compartment 225 and the suction control compartment 238 is arranged close to one of the distal corners of the suction control compartment 238 leading into the suction compartment 225 in a central region thereof, close to the corresponding lateral border.

In the assembled state, the top surface 221 of the valve body 202 is covered by a top cover 235 made from PVC, matching the footprint of the valve body 202. The top cover 235 features a keyhole shaped opening 236 which in the assembled state connects the suction control compartment 238 to the exterior. It further comprises a control surface 233 made from a flexible material which in the assembled state is arranged in the region of the valve compartment 223. The base surface 221 is covered by a base cover 234 matching the footprint of the valve body 202 and having a continuous surface. The length of the valve body 202 with attached top and base covers 234, 235 is about 30 mm, the width is about 25 mm and the thickness is about 11 mm.

The FIGS. 9A, 9B are schematical illustrations of the valves in their suction and irrigation position, respectively. They show the same cross-section as FIG. 8B.

A ball 240 made from a ferromagnetic material (such as stainless steel) is inserted into the valve compartment 223. A permanently magnetized ring 241 is accommodated in the mouth region of the irrigation bore 227 leading into the valve compartment 223. In order to accommodate the ring 241, in its mouth region the diameter of the irrigation bore 227 has a correspondingly larger diameter. The inside diameter of the magnetized ring 241 corresponds to the diameter of the main portion of the irrigation bore 227.

Usually, the ball 240 is attracted to the permanently magnetized ring 241 and thereby closes the connection of the valve compartment 223 to the irrigation bore 227, i.e. the irrigation passage is closed. The inner edge of the permanently magnetized ring 241 constitutes a valve seat for the ball 240. At the same time, the suction compartment 225 is connected to the valve compartment 223 by the connection bore 232, i.e. the suction passage is open. In the region of the valve compartment 223, at least in the central region thereof, the top cover 235 is made from a flexible material which allows for pushing the ball 240 along a vertical path in order to displace the ball 240 to block the connection bore 232, the inner edge of the connection bore 132 constituting another valve seat for the ball 240. In this second position, the suction passage is closed but the irrigation passage is open.

The handling of the suction and irrigation device 201 according to the second embodiment essentially corresponds to the handling of the first embodiment explained before. As mentioned, as long as the flexible region of the top cover 235 is not pushed, the suction and irrigation device 201 is in a first initial position, wherein the suction passage is open and the irrigation passage is closed. The suction/irrigation tube connected to the exit bore 228 can be placed at an operation area, wherein liquid is supposed to be drawn off. The valve body 202 is held between a thumb and a forefinger, while the thumb closes the opening 236 to the suction control compartment 238 connected to the suction compartment 225. A suction apparatus is connected to the suction bore 226 by means of a suitable connector. The suction apparatus applies a suction force at the tip of the suction/irrigation tube. Since the opening 236 is closed full power of the suction force is generated at the tip and a strong suction effect acts on the liquid in the operation area.

To reduce the suction effect in the suction/irrigation tube, the opening 236 in the top cover 235 of the valve body 202 can be partially or completely opened by lifting the thumb. Such the suction compartment 225 is open to the atmosphere and the suction apparatus partly sucks air from the atmosphere. Therefore the suction effect at the tip of the suction/irrigation tube is reduced.

By pushing the control surface 233 of the top cover 235, e.g. with the thumb, the suction and irrigation device may be brought to the second position, where the suction passage is closed and the irrigation passage is opened. The irrigation bore 227 is connected to an irrigation apparatus by a suitable connector, the apparatus providing irrigation fluid for the suction and irrigation device. The irrigation fluid flows through the open irrigation passage and the suction/irrigation tube to the operation area and flushes the area. By releasing the control surface 233 it will reset to its initial state due to the elasticity of the material and the magnetic forces attracting the ball 240 will move the ball 240 to the magnetic ring 241, thereby closing the irrigation bore 227.

List of reference symbols

| | |
|---|---|
| 1 | Suction and irrigation device |
| 2 | Valve body |
| 3 | Irrigation pipe |
| 4 | Suction pipe |
| 5 | Suction/irrigation tube |
| 6 | Push surface |
| 7 | Irrigation passage |
| 8 | Suction passage |
| 9 | First connector |
| 10 | Second connector |
| 11 | Distal connector |
| 12 | Partition wall |
| 13 | Intermediate cavity |
| 14 | Exit channel |
| 15 | First closing ledge |
| 16 | Second closing ledge |
| 17 | Lamella |
| 18 | Lamella |
| 19 | Opening |
| 102 | Valve body |
| 121 | Base surface |
| 122 | Top surface |
| 123 | Valve compartment |
| 124 | Irrigation compartment |
| 125 | Suction compartment |
| 126 | Suction bore |
| 127 | Irrigation bore |
| 128 | Exit bore |
| 129 | Bore |
| 130 | Connection bore |
| 131 | Bore |
| 132 | Connection bore |
| 135 | Cover |
| 136 | Opening |
| 140 | Ball |
| 141 | Ring |
| 142 | Pushing rod |
| 143 | Plug |
| 201 | Suction and irrigation device |
| 202 | Valve body |
| 221 | Base surface |
| 222 | Top surface |
| 223 | Valve compartment |
| 225 | Suction compartment |
| 226 | Suction bore |
| 227 | Irrigation bore |
| 228 | Exit bore |
| 232 | Connection bore |
| 233 | Control surface |
| 234 | Base cover |
| 235 | Top cover |
| 236 | Opening |
| 237 | Connection bore |
| 238 | Suction control compartment |
| 240 | Ball |
| 241 | Ring |

While the system, apparatus, process and method herein described constitute preferred embodiments of this invention, it is to be understood that the invention is not limited to this precise system, apparatus, process and method, and that changes may be made therein without departing from the scope of the invention which is defined in the appended claims.

What is claimed is:

1. A suction and irrigation device for medical use comprising:
a valve body comprising an irrigation passage and a suction passage, whereas at least one irrigation pipe and at least one suction pipe are connectable to the irrigation passage and the suction passage, respectively, by connections at a proximal side of the valve body and whereas a suction/irrigation tube is connectable to the irrigation passage and the suction passage at a distal side of the valve body by at least one further connection,
a valve assembly within the valve body, which in a first position opens the suction passage and closes the irrigation passage and in a second position closes the suction passage and opens the irrigation passage; and
at least one push element which interacts with at least one movable element of the valve assembly, whereas the movable element is movable by mechanical force acted upon the push element such that the push element activates the valve assembly from one position to the other position, wherein in a state where no mechanical force is acted upon the push element, the movable element is held by elastic forces in a position where the irrigation passage is closed and where the suction passage is open,
wherein the movable element is ferromagnetic and the irrigation passage is provided with at least one ferromagnetic element, and wherein the at least one of the ferromagnetic element and the movable element is permanently magnetized such that the elastic forces are exerted by a magnetic field, and in that upon action of a mechanical force upon the push element the movable element is moved in a direction parallel to a wall including the irrigation passage until it covers the suction passage.

2. (canceled)

3. The suction and irrigation device according to claim 1, wherein the valve assembly comprises a first valve within the suction passage and a second valve within the irrigation passage, which are commonly activated by the push element.

4-17. (canceled)

18. The suction and irrigation device according to claim 3, wherein the movable element is ball shaped.

19. The suction and irrigation device according to claim 3, wherein a diameter of the ring surrounding the passage is in the range of 0.5-2 times the diameter of the movable element.

20. (canceled)

21. The suction and irrigation device according to claim 1, wherein the valve body is carried by a strong housing comprising at least one opening to activate the at least one push element.

22. The suction and irrigation device according to claim 1, wherein an intermediate cavity is arranged between the suction passage and the irrigation passage within the valve body next to the valve assembly.

23. The suction and irrigation device according to claim 1, wherein the push element comprises an opening to the suction passage.

24. The suction and irrigation device according to claim 1, wherein the diameter of a ring surrounding the passage is in the range of 0.8-1.2 times the diameter of the movable element.

25. The suction and irrigation device according to claim 1, wherein the ferromagnetic element is a ring surrounding the passage.
